(19) United States
(12) Patent Application Publication
(10) Pub. No.: US 2008/0127555 A1
(43) Pub. Date: Jun. 5, 2008

(54) Gas Odorant

(76) Inventors: Philip Kraft, Duebendorf (CH); Urs Mueller, Duebendorf (CH); John Norman Short, L'Isle Adam (FR)

Correspondence Address: NORRIS, MCLAUGHLIN & MARCUS, 875 THIRD AVE, 18TH FLOOR, NEW YORK, NY, 10022, US

(21) Appl. No.: 11/795,786

(22) PCT Filed: Nov. 8, 2005
(86) PCT No.: PCT/CH05/00655

§ 371 Date: Oct. 12, 2007

(30) Foreign Application Priority Data
Nov. 9, 2004 (EP) 04300771.5
Mar. 2, 2005 (EP) 05290717.7

(51) Int. Cl. C10L 3/00; C07C 11/30

(52) U.S. Cl. 48/197 R; 44/628

(57) ABSTRACT

The present invention refers to the use of C7 to C9 cycloalkynes as gas odorant, to a process of odorising gas and to fuel gas comprising it.

The present invention relates to the use of C7 to C9 cycloalkynes as gas odorants, to a process of odorising gas and to fuel gas comprising them.

Because of their origin and their relative high degree of purity, fuel gases are substantially odorless. If leakages are not noticed in good time, explosive mixtures with a high hazard potential may be quickly formed. For safety reasons, gas is therefore odorized by adding odorants. For example, tetrahydrothiophene (THT) is used throughout the European Union area as a city gas odorant, usually as the sole odorant. In North America, tertiary butyl mercaptan is widely used as the principal odorant, often in association with other alkyl mercaptans and various sulphides and disulphides. For liquid petroleum gas, for example, ethyl mercaptan is used as an odorant. These compounds are particularly well suited for use as gas odorants because of their very powerful) unpleasant and distinctive odours. Furthermore, because they have been used for a very long time, they are nowadays universally associated with combustible gases and thus meet the requirement for a distinct and well recognized warning of a gas leakage. However, with regard to environmental aspects, sulfur compounds are less suitable because sulfur dioxide is formed during the combustion of such odorized gases. Furthermore, sulfur-based compounds are known to poison electrode catalysts of fuel cells when converted into H₂S, which results in severe degradation of the performance of the fuel cell.

To be highly suitable as gas odorant, a compound or a mixture of compounds, i.e. a composition, has to fulfill a number of requirements. In particular, to avoid the danger of confusion, the odour of the gas odorant needs to:
be distinctive enough that there is very little risk that it will be identified as any other smell. Ideally it should be immediately associated with the odour of current gas odorants, because their broad use make the odour of gas easily recognizable to the majority of users.
have a detection threshold several orders of magnitude lower than the explosive limit of the fuel gas in order that everybody with an average sense of smell and average physiological conditions is able to detect the odor.

Furthermore, the gas odorant has to be stable under the storage and transport conditions of the fuel gas.

Several attempts have been made to replace or at least reduce the use of sulfur compounds as odorant in fuel gas. For example, DE-A 19837066 describes the use of a mixture of acrylic alkyl esters and nitrogen compounds. A fuel gas odorant comprising an alkyne such as butyne-1, vinylacetylene and hexyne, and at least two compounds selected from methyl acrylate, ethyl acrylate, methyl methacrylate, allyl methacrylate, ethyl propionate, methyl n-butyrate, and methyl isobutyrate is disclosed in JP-A-55-104393 (abstract). The problem with acrylic alkyl esters is that their odor notes are very similar to, for example, certain acrylic plastics and paints.

Accordingly, there is an ongoing demand to find alternative odorants which are particularly suitable as gas odorant. Surprisingly, it has been found that certain cycloalkynes are particularly suitable as odorant for fuel gas.

Thus the present invention refers in one of its aspects to the use as fuel gas odorant of cycloalkynes of formula (I)

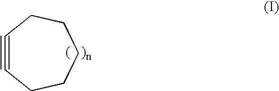

wherein n is 1, 2, or 3, and up to 2 hydrogen atoms, i.e. none, 1, or 2 hydrogen atom(s), are substituted by a methyl group.

Particularly preferred as fuel gas odorant is cyclooctyne.

The term “gas odorant” as used within the meaning of this invention may refer to both a single odorous compound and a mixture of such odorous compounds.

Fuel gases are in general used for generating electrical power by combustion in power stations, or used in buildings for heating, illuminating and cooking processes. They can also be used to generate hydrogen gas for use in hydrogen fuel cells by a process commonly known as “reforming”. The term “fuel gas” as used within the meaning of this application stands for any combustible hydrogen or hydrocarbon gas used as a primary or secondary energy source. They are in gaseous form at normal atmospheric temperature and pressure (25° C.; 1000 mbar) but may also be processed in their liquid form for convenience of transport and storage. Fuel gases encompass, but are not limited to, the terms: city or town gas, natural gas including its liquefied form, liquid petroleum gas (LPG, which is a mixture of alkanes separated from petrol and consisting essentially of butane and propane), and hydrogen gas. Alkynes, such as acetylene, are also suitable as fuel gas. Oxygenated hydrocarbons, such as dimethyl ether, that can be used as a source of energy by combustion or to generate hydrogen for fuel cells, also belong to the class of fuel gases within the meaning of this application.

Surprisingly it was found that the odor of the gas odorant of the present invention is even better when the cycloalkyne of formula (I) is combined with an alkyne of formula (I)

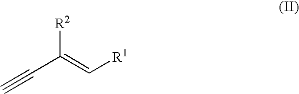

wherein
R¹ is hydrogen, methoxy or ethoxy; and
R² is hydrogen or methyl.

Particular preferred compounds of formula (II) may be selected from the list consisting of 1-methoxy-buten-3-yne, and 2-methyl-1-buten-3-yne.

Thus, in a further aspect the present invention refers to the use as fuel gas odorant of a composition comprising:
a) at lease one cycloalkyne of formula (I); and
b) at least one alkyne of formula (II).

In another embodiment the compounds of the present invention may be used in combination with known gas odorants, i.e. with sulfur compounds and sulfur-free compounds. Particular preferred is the combination with sulfur-free gas odorants, for example pyrazines. Preferably the fuel gas odorant comprises up to 10 weight %, more preferably 0.1 to 5.5 weight % of a pyrazine, based on the total amount of gas odorant. If combined with sulfur compounds the fuel gas odorant preferably comprises up to 60 weight %, more preferably 1 to 30 weight %, for example, 1 to 10 weight %, of at least one sulfur compound or a mixture thereof, based on the total amount of gas odorant.

Suitable pyrazines include but are not limited to methyl ethyl pyrazine, methoxy isobutyl pyrazine, and methoxy methylpyrazine. Further suitable pyrazines are disclosed in JP-A-08-60167, which is incorporated by reference. By admixing the compounds of the present invention together with a smaller amount of pyrazine even better results can be achieved.

Suitable sulfur compounds include but are not limited to compounds selected from the group consisting of C₁-C₄ alkyl mercaptane, e.g., tert.-butyl mercaptan and ethyl mercaptan, aryl mercaptanes, e.g. benzyl mercaptan, organic sulfides and disulfides, e.g. dimethyl sulfide and ethyl methyl sulfide, and tetrahydrothiophene and their derivatives.

Thus a further aspect of the present invention is a gas odorant comprising:
a) 5 to 100 weight %, preferably at least 10 weight % of at least one cycloalkyne of formula (I);
b) up to 95 weight % (i.e. 0-95 weight %), preferably up to 90 weight % of at least one alkyne of formula (II); and
c) up to 10 weight % (i.e. 0-10 weight %), preferably up to 5 weight % of a pyrazine; and
d) up to 60 weight % (i.e. 0-60 weight %), preferably up to 30 weight % of at least one sulfur compound.

Other excipients such as antioxidants may also be added, either to the odorant or directly to the odorized fuel gas. Suitable antioxidants include but are not limited to tert butylhdroxyanisole, 2,5-di-tert-butyl-phenol (Ionol), hydroquinone monomethyl ether and α-tocopherol, 2,6-di-tert-butyl para cresol and tert-butyl hydroxy toluene.

A further aspect of the present invention is a fuel gas comprising a gas odorant comprising
a) 5 to 100 weight %, preferably at least 10 weight % of at least one cycloalkyne of formula (I);
b) up to 95 weight % (i.e. 0-95 weight %), preferably up to 90 weight % of at least one alkyne of formula (II); and
c) up to 10 weight % (i.e. 0-10 weight %), preferably up to 5 weight % of a pyrazine, preferably selected from the group consisting of methyl ethyl pyrazine, methoxy isobutyl pyrazine and methoxy methylpyrazine
d) up to 60 weight % (i.e. 0-60 weight %), preferably up to 30 weight % of a sulfur compound, preferably selected from the group consisting of tert.-butyl mercaptan, ethyl mercaptan, benzyl mercaptan, dimethyl sulfide, ethyl methyl sulfide and tetrahydrothiophene.

The dosage of the odorant in the gas mainly depends on the composition of the odorant and may vary from 1 to about 100 ppm, preferably between 5 and 50 ppm, more preferably between 20 and 40 ppm.

Furthermore the present invention refers to a method of odorizing fuel gas comprising the incorporation as odorant of an effective amount of at least one cycloalkyne as hereinabove described.

The gas odorants of the present invention are liquids at room temperature and thus, both the preparation of an odorant composition if required and the admixing of the odorant/odorant composition to the fuel gas is not critical, methods and equipments known to the person skilled in the art may be used.

The invention is now further described with reference to the following non-limiting examples.

### EXAMPLE 1

### Gas Odorant Compositions

Table 1 shows preferred gas odorants A to K according to the present invention and gas odorants 1 to 3 as reference.

TABLE 1

| odorant | CYC | MET | MEP | MMP | other compounds |
|---|---|---|---|---|---|
| A | 10 | 90 | | | |
| B | 10 | | | | 90 (propyne) |
| C | 10 | 89 | 1 | | |
| D | 10 | 89.5 | | 0.5 | |
| E | 100 | | | | |
| F | 10 | | | | 90 (2ME) |
| G | | 100 | | | |
| H | 8 | 72 | | | 20 (TBM) |
| I | 8 | 72 | | | 20(THT) |
| J | 8 | 72 | | | 14 (DMS), 6 (TBM) |
| K | 80 | | | | 20 (TBM) |
| 1 | | | | | 100 (THT) |
| 2* | | | 2.5 | | 60 (EAC), 37.5 (MAC) |
| 3** | | | | | Labogaz |

% by weight
*Gasodor S-FREE ® as disclosed in Gas und Wasserfach, Gas, Erdgas 142/11 732, 779-780, 782-784 (November 2001)
**commercial propane/butane gas with a mixture of sulfur compounds as gas odorant. Sold in France as Campingaz ™.
The following abbreviations are used in Table 1:
CYC: Cylcooctyne
MET: 1-Methoxy-buten-3-yne
2ME: 2-Methyl-1-buten-3-yne
MEP: methyl ethyl pyrazine
MMP: methoxy methyl pyrazine
TBM: tert.-butyl mercaptan
THT: tetrahydrothiophene
DMS: dimethylsulphide
EAC: ethyl acrylate
MAC: methyl acrylate

### EXAMPLE 2

### Comparison Study to Known Gas Odorants

Isobutane gas aerosol comprising the odorants A, B, E-I, 1, 2 or 3 (Example 1, Table 1) at a dosage of 40 ppm were prepared. The aerosol was sprayed into a booth of 7 m³ and sniffed through a small port in the door of the booth by a panel of 30 individuals, who are familiar with the smell of gas. They had been asked to evaluate the smell, having the smell of gas in mind, on a 4 point scale (4=totally agree (i.e. equal to the odour of gas), 3=mostly agree, 2=mostly disagree, and 1=totally disagree). The odorants were tested blind and the panellists were not aware of the composition. The results are listed in Table 2.

TABLE 2

| odorant | Similarity |
|---|---|
| A | 2.56 |
| B | 1.63 |
| E | 2.33 |
| F | 1.88 |
| G | 2.33 |
| H | 2.41 |
| I | 3.53 |
| J | 3.06 |
| 1 | 3.88 |
| 2 | 1.84 |
| 3 | 3.90 |

1. A fuel gas odorant which is a cycloalkyne of formula (I) 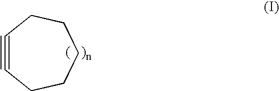
wherein n is 1, 2, or 3, and
up to 2 hydrogen atoms are substituted by a methyl group.

2. (canceled)

3. A fuel gas odorant comprising a cycloalkyne of formula (I) according to claim 1 with an alkyne of formula (II) 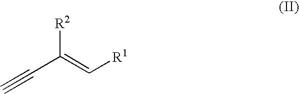
wherein
R¹ is hydrogen, methoxy or ethoxy; and
R² is hydrogen or methyl.

4. A fuel gas odorant according to claim 1 which further comprises a pyrazine and/or sulfur compound.

5. A gas odorant comprising
a) 5 to 100 weight %, of at least one cycloalkyne of formula (I) 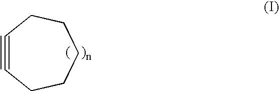
wherein n is 1, 2, or 3, and
up to 2 hydrogen atoms are substituted by a methyl group;
b) up to 95 weight % of at least one alkyne of formula (II) 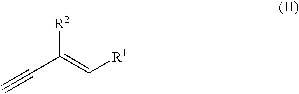
wherein
R¹ is hydrogen, methoxy or ethoxy; and
R² is hydrogen or methyl; and
c) up to 10 weight % of a pyrazine
d) up to 60 weight % of a sulfur compound.

6. A fuel gas comprising a gas odorant according to claim 5.

7. A method of odorizing fuel gas comprising the incorporation of an effective amount of at least one cycloalkyne of formula (I) 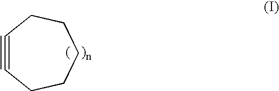
wherein n is 1, 2, or 3, and
up to 2 hydrogen atoms are substituted by a methyl group, to the fuel gas.

8. A method of odorizing fuel gas comprising the incorporation of an effective amount of gas odorant according to claim 5 to the fuel gas.

9. A gas odorant according to claim 5 comprising
a) 5 to 100 weight % of at least one cycloalkyne of formula (I) 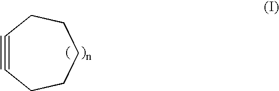
wherein n is 1, 2, or 3, and
up to 2 hydrogen atoms are substituted by a methyl group;
b) up to 95 weight % of at least one alkyne of formula (II) 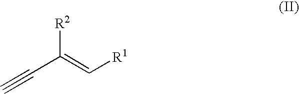
wherein
R¹ is hydrogen, methoxy or ethoxy; and
R² is hydrogen or methyl; and
c) a pyrazine present in an amount of up to 10 weight %; or,
d) a sulfur compound present in an amount of up to 60 weight %; or,
e) a pyrazine present in an amount of up to 10 weight % with a sulfur compound present in an amount of up to 60 weight %.
